(19) United States
(12) Patent Application Publication
(10) Pub. No.: US 2025/0134543 A1
(43) Pub. Date: May 1, 2025

(54) COUPLING PROXIMAL AND DISTAL PORTIONS OF A MEDICAL DEVICE

(71) Applicant: Boston Scientific Scimed, Inc., Maple Grove, MN (US)

(72) Inventors: Ryan V. WALES, Northborough, MA (US); Scott E. BRECHBIEL, Burlington, MA (US); Paul SMITH, Smithfield, RI (US); Kurt Nicholas ROBAKIEWICZ, Upton, MA (US); Rosangel Aimee RAMOS ESPINOZA, Allston, MA (US); John B. GOLDEN, Norton, MA (US)

(73) Assignee: Boston Scientific Scimed, Inc., Maple Grove, MN (US)

(21) Appl. No.: 18/925,468

(22) Filed: Oct. 24, 2024

Related U.S. Application Data
(60) Provisional application No. 63/593,337, filed on Oct. 26, 2023.

(51) Int. Cl. A61B 17/29; A61B 17/00

(52) U.S. Cl. CPC A61B 17/2909; A61B 2017/00473; A61B 2017/2924; A61B 2017/2947

(57) ABSTRACT

A medical device may comprise a handle, including a main body and an actuator. The main body may include a first portion and a second portion. The main body may have an open configuration and a closed configuration. The actuator may include a first portion and a second portion. The actuator may have an open configuration and a closed configuration. The actuator may translate relative to the main body in the closed configurations of the main body and the actuator.

### CROSS-REFERENCE TO RELATED APPLICATIONS

This application claims the benefit of priority of U.S. Provisional Patent Application No. 63/593,337, filed on Oct. 26, 2023, the entirety of which is incorporated herein by reference.

### TECHNICAL FIELD

The disclosure relates generally to a coupling for medical devices. More specifically, aspects of the disclosure pertain to a releasable coupling for attaching a proximal portion of a medical device to a distal portion of the medical device.

### BACKGROUND

Single-use medical devices can result in a large amount of waste, burdening the environment. For example, many endoscopic devices, including hemostasis clips and biopsy forceps, are currently single-use devices. Such devices often include a handle, a shaft, and a distal tip with an end effector. The wire (attaching the handle and the end effector) and the shaft are inserted in the body to perform a procedure at a tissue site (e.g., obtain a tissue sample) while the handle remains outside the body. Disposal of the entire device creates large amounts of waste and environmental burden. Therefore, there is a need for an apparatus that results in less waste and environmental burden. The apparatus and methods described herein may alleviate this deficiency and one or more other deficiencies in the art. However, the attached claims specify the scope of this disclosure rather than the ability to solve any specific problem.

### SUMMARY

This disclosure includes devices and methods that allow for releasably coupling a proximal component of a medical device (including a handle) to a distal component of the medical device (including a portion inserted in the body during use). Such devices and methods permit, for example, the reuse of the proximal component with other distal components, resulting in less waste. Each of the aspects disclosed herein may include one or more of the features described in connection with any other disclosed aspects.

In an example, a medical device may comprise: a handle including a main body and an actuator. The main body may include a first portion and a second portion, and the main body may have an open configuration and a closed configuration. The actuator may include a first portion and a second portion. The actuator may have an open configuration and a closed configuration. The actuator may translate relative to the main body in the closed configurations of the main body and the actuator.

Any examples described herein may have any of these features alone or in any combination. The first portion of the main body may pivot relative to the second portion of the main body to transition from the open configuration to the closed configuration. The first portion and the second portion of the main body may include a first rail and a second rail, wherein each of the first rail and the second rail may extend longitudinally in a proximal to distal direction and may define a slot therebetween. The first portion of the main body and the second portion of the main body may include a wire channel extending longitudinally. The first portion of the actuator and the second portion of the actuator may include indentations for placing over the first rail and the second rail. The first portion of the actuator may pivot relative to the second portion of the actuator to transition from the open configuration to the closed configuration. The handle may include a distal tip, wherein the distal tip includes a first portion and a second portion, and the distal tip has an open configuration and a closed configuration. The first portion of the distal tip and the second portion of the distal tip may define a proximal slot communicating with a distal slot having a cross-section that is wider than a cross-section of the proximal slot. The proximal slot and the distal slot of each of the first portion of the distal tip and the second portion of the distal tip may respectively receive a neck portion and a keyed portion of each of a corresponding first portion of the main body and the second portion of the main body. The keyed portion may have a cross-sectional shape to match a shape of a corresponding distal slot. The first portion of the distal tip may be pivotally connected to the second portion of the distal tip to transition between the open configuration and the closed configuration of the distal tip. The shaft may extend from a distal end of the handle and radially surrounds a wire. The shaft may include a shaft crimp, the wire may include a wire crimp, and the wire crimp may be positionable in the actuator, and the shaft crimp may be positionable in the distal tip.

The first end of the actuator may include a first wire opening and a first crimp slot, and a second end of the actuator may include a second wire opening and a second crimp slot. The shaft may include a proximal portion that is flared outwardly, and the medical device may include a collar surrounding the flared proximal portion of the shaft, wherein the distal tip may include a slot, and wherein the collar may be receivable into the slot. The actuator may include an opening extending radially through the actuator, wherein the actuator may include a knob that rotates within the opening. The knob may define an opening therein to receive a wire.

In another example, a medical device may comprise a proximal component. The proximal component may include a handle. The handle may include a main body and an actuator. The main body may include a first portion and a second portion that may be movable relative to the first portion of the main body to transition between an open configuration and a closed configuration of the main body. The actuator may include a first portion and a second portion that may be movable relative to the first portion of actuator to transition between an open configuration and a closed configuration of the main body. The actuator may translate relative to the main body in the closed configurations of the main body and the actuator. The medical device may further comprise a distal component. The distal component may include a shaft and a wire and the distal component may be releasably coupled to the proximal component.

Any of the devices disclosed herein may include any of the following features in any combination. The proximal component may include a distal tip. The distal tip may include a first portion and a second portion that may be movable relative to the first portion of the distal tip and may transition between an open configuration and a closed configuration of the distal tip. The shaft may extend from a distal end of the proximal component and radially surrounds the wire. The shaft may include a portion that is flared outwardly, and the medical device may further comprise a collar that surrounds the flared portion of the shaft, the distal tip may include a slot, and the collar of the shaft may be receivable into the slot. The actuator may include an opening that may extend radially through the actuator. The actuator may include a knob that rotates within the opening. The knob may define an opening therein to receive a wire.

A medical method may comprise: placing a first actuator portion onto a first main body portion, inserting a wire into a wire opening of the first actuator portion and a wire channel of the first main body portion, closing a first main body portion with a second main body portion, the first main body portion and the second main body portion are configured to be closed by pivoting the first main body portion relative to the second main body portion; and closing the first actuator portion with a second actuator portion, wherein the first actuator portion and the second actuator portion are configured to be closed by pivoting the first actuator portion relative to the second actuator portion.

It may be understood that the preceding general and detailed descriptions are exemplary and explanatory only and are not restrictive of the invention, as claimed. As used herein, the terms “comprises,” “comprising,” “has,” “having,” “includes,” “including,” or any other variation thereof are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example” rather than “ideal.” The term “distal” refers to a direction away from an operator/toward a treatment site, and the term “proximal” refers to a direction toward an operator. The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate examples of this disclosure and, together with the description, explain the principles of the disclosure.

FIG. 1 illustrates a perspective view of an exemplary medical device, including an enlarged view of a distal portion of the medical device, according to an embodiment;

FIG. 2 illustrates a partially cutaway view of the proximal portion of the medical device of FIG. 1;

FIG. 3 illustrates a perspective view of components of the proximal assembly of the medical device of FIG. 1 in an open configuration (and separated for ease of view) and proximal portions of a wire and a shaft for coupling to the proximal assembly;

FIG. 4 illustrates a perspective view of half of the handle tip of the proximal assembly of FIG. 3;

FIG. 5 illustrates a perspective view of the proximal assembly of the medical device of FIG. 1 in a closed, connected configuration;

FIG. 6 illustrates a perspective view of another embodiment of half of a handle tip assembly;

FIGS. 7A-7B illustrate side views of another embodiment of half of a spool assembly; and

FIG. 8 illustrates a cross-sectional side view of another embodiment of half of a spool assembly.

### DETAILED DESCRIPTION

Reference is now made in detail to examples of this disclosure, aspects of which are illustrated in the accompanying drawings. The same reference numbers will be used throughout the drawings to refer to the same or like parts wherever possible.

Embodiments of this disclosure include a medical device with a proximal component that releasably couples to a distal component. The proximal component may include a handle having various parts with open and closed configurations. The distal component may include a shaft, an actuation wire, and an end effector.

Before use of the medical device, the handle may be in an open configuration. In this configuration, the proximal portions of the wire and the shaft may releasably couple to the handle, and then the handle may be transitioned to a closed configuration for securing the distal component to the handle. After a procedure, the user may transition the handle back to the open configuration, and the wire's proximal portion and the shaft's proximal portion may be decoupled from the handle. In this way, the distal component, which contacts the patient during use, may be disposed of, and the proximal component may be cleaned and reused with a second distal component in a subsequent procedure.

Such embodiments of a medical device may result in reduced waste and reduced overall cost of the medical device (fewer handles needed, less packaging, etc.). Such embodiments may also result in a universal proximal component (including a handle) that may be used with numerous distal components (including different end effectors).

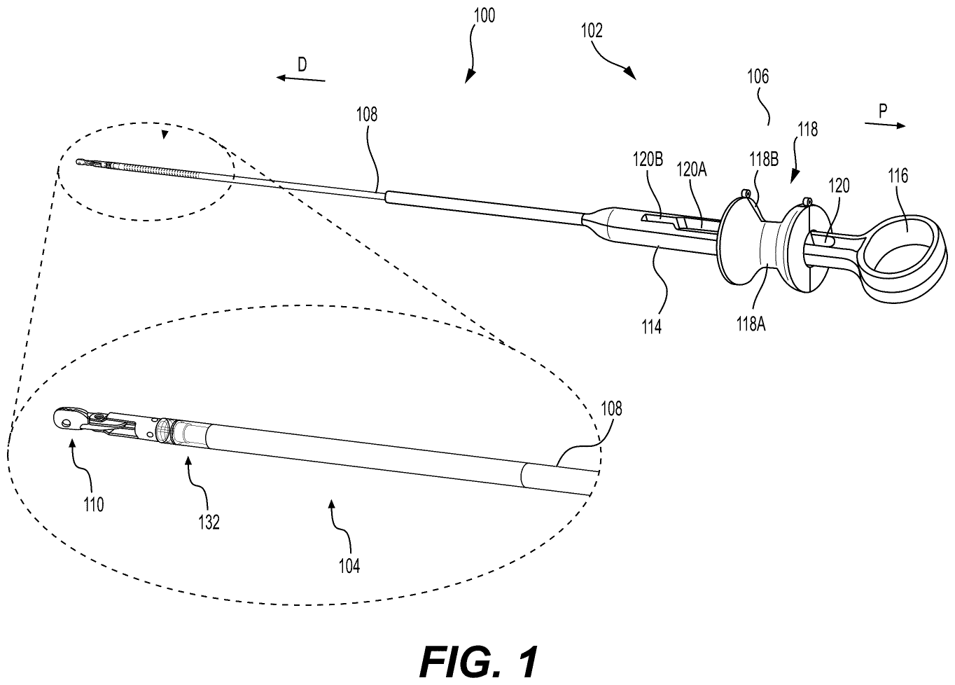

FIG. 1 illustrates a medical device 100, including a proximal portion 102, a shaft 108, and a distal portion 104. FIG. 1 includes an enlarged view of distal portion 104. The shaft 108 may extend from the distal portion 104 to the proximal portion 102. The shaft 108 may include or otherwise radially surround a wire 138, such as a pull or actuation wire. The wire 138, the shaft 108, and the distal portion 104 may be considered parts of the distal component of the medical device 100. The wire 138 may extend from the handle 106 through the shaft 108, and the wire 138 may be movable (e.g., within and relative to the shaft 108) via manipulation of one or more portions of the handle 106 to control one or more aspects of the distal portion 104 of the medical device 100. For example, as discussed below, the end effector 110 may be at a distal end of the medical device 100, at the distal portion 104.

The handle 106 includes a main body 114 that may include a ring 116 (e.g., a thumb ring) at the proximal end of the main body 114. The handle 106 also includes a first movable member, or an actuator that may be in the form of a spool 118. The spool 118 may include an indented portion 118A (e.g., with a relatively smaller lateral cross-section) and one or more (e.g., two) ridged or extended portions 118B (e.g., with a relatively larger lateral cross-section). In these aspects, the indented portion 118A may receive one or more of the user's fingers, such that movement of the user's finger(s) controls the movement of the spool 118. The main body 114 may include a slot 120, extending longitudinally through a portion of main body 114, for example, from a position spaced distally from the ring 116. A portion of the spool 118 may extend into a portion of the slot 120, such that the spool 118 is movable (e.g., longitudinally movable distally and/or proximally) along the slot 120. In these aspects, the slot 120 may define a range of movement for the spool 118. The spool 118 may be movable within the slot 120, for example proximally and/or distally, to control one or more aspects of the end effector 110.

Furthermore, the slot 120 may include a wide portion 120A and a tapered or narrow portion 120B. As discussed below, the wide portion 120A may be proximal to the narrow portion 120B, and the transition between the wide portion 120A and the narrow portion 120B may support or otherwise provide a stop surface for a biasing element 122.

Medical device 100 includes the end effector 110 at the distal portion 104. The end effector 110 may be a forceps, including two jaws. Nevertheless, this disclosure is not so limited, and the end effector 110 may be any type of end effector, instrument, tool, or other device (e.g., grasper, snare, basket, hemostatic clip, stapler, ablation device, tome, suturing device, needle, knife, etc.). The end effector 110 may be coupled to the shaft 108 at the distal end, for example at a coupling portion 132. The coupling portion 132 may include various coupling apparatus, for example, a clevis, a bushing, or any other structure, including conventional structures for coupling the end effector 110 to the shaft 108 and the actuation wire 138.

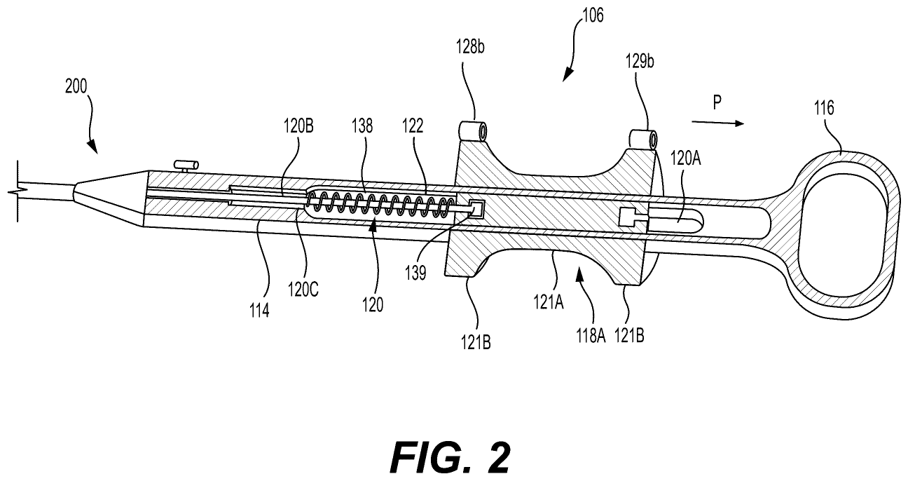

FIG. 2 is a partially cutaway view of the proximal portion 102 of the medical device 100 of FIG. 1, including the handle 106. As discussed, the handle 106 includes the spool 118, which is movable relative to main body 114, for example, within a portion of the slot 120. The handle 106 additionally includes a spring or biasing element 122. The biasing element 122 is positioned within the slot 120, and specifically the wide portion 120A, and may be stopped (rest against) a distal stop surface 120C at the transition from the wide portion 120A to the narrow portion 120B. The biasing element 122 may bias spool 118 proximally. For example, the biasing element 122 may compress as the spool 118 is moved distally, and the biasing element 122 may urge the spool 118 proximally once the distal force on the spool 118 is removed. In addition, the movement of the spool 118 may control the actuation of the end effector 110. For example, longitudinal movement of the spool 118 in a first direction (e.g., in the distal direction) may actuate the end effector 110.

As discussed, before using the medical device 100, the handle 106 may be in an open configuration. In an open configuration, the proximal portion of the wire 138 and the shaft 108 may releasably couple to the handle 106. The handle 106 then may be transitioned to a closed configuration to secure the wire 138 and the shaft 108 to the handle 106, so that the actuation of the handle 106 may actuate the end effector 110. After use of the medical device 100, the wire 138 and the shaft 108 may be decoupled from the handle 106. This way, the distal component of the medical device 100, including the distal portion 104, the shaft 108, and the wire 138, may be disposed of, and the proximal component (including the handle 106) may be reused.

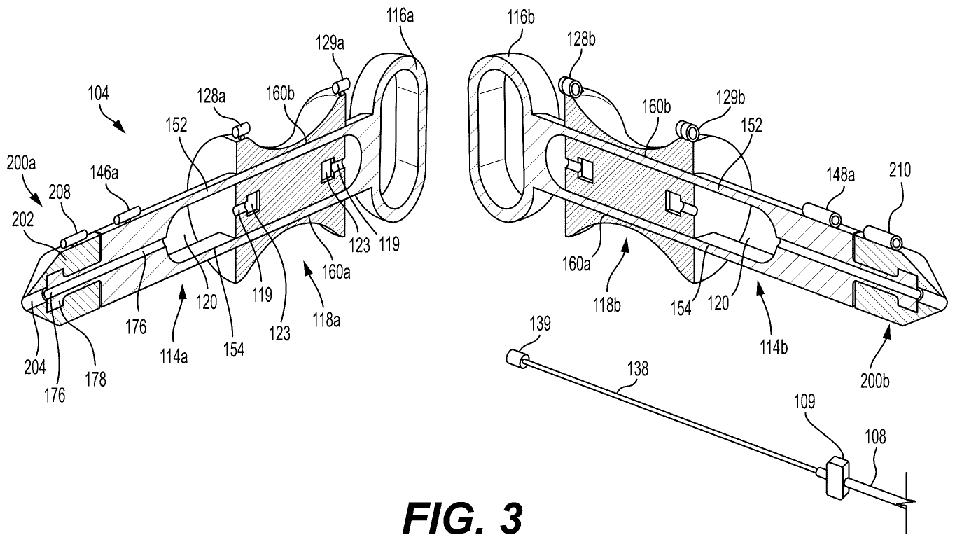

FIG. 3 illustrates a perspective view of components of the handle 106 in an open configuration (and separated for ease of view) and proximal portions of the wire 138 and the shaft 108 for coupling to the handle 106. As discussed, the handle 106 may have three main components: the main body 114, a distal handle tip 200, and the spool 118. First, the main body 114 is described, followed by the spool 118 and the distal tip 200.

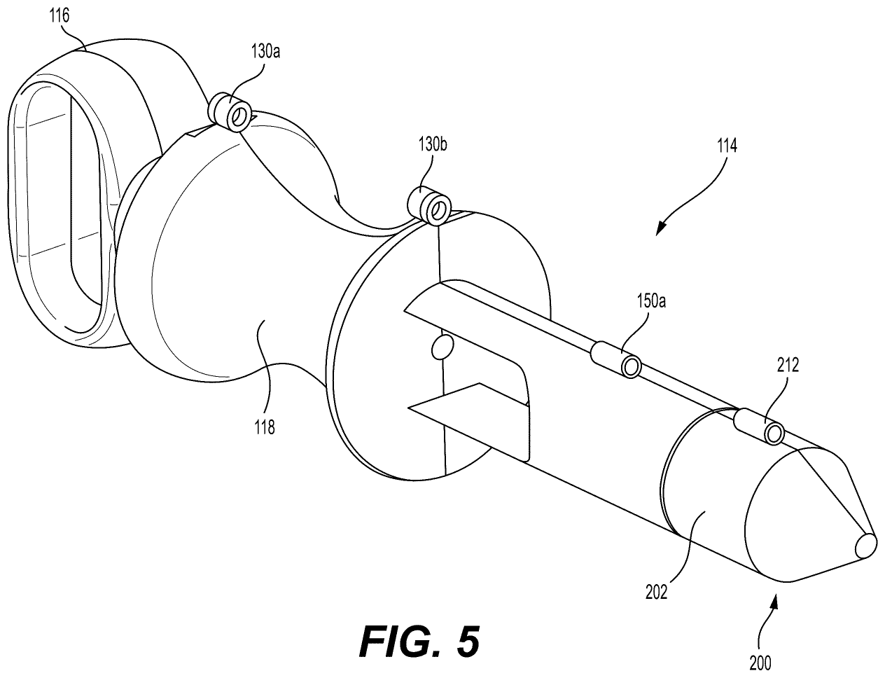

The main body 114 may include two halves, 114a and 114b. The division of the main body 114 may be along a longitudinal plane at a center of the main body 114. As shown in FIG. 3, halves 114a and 114b may be in an open, separated configuration. FIG. 3 shows the halves 114a, 114b completely unattached for ease of view of the details of the halves 114a, 114b, but in embodiments, the halves 114a, 114b may be fixed together in a manner that permits opening and closing of the main body 114, such as by way of hinges to be described. The first main body half 114a and the second main body half 114b may be identical except for the coupling members of the hinges described below. As discussed, the main body 114 may include the ring 116 at the proximal-most end. Two identical ring halves, 116a and 116b form the ring 116 in a closed configuration shown in FIG. 5.

The main body half 114a may include a first rail 152 and a second rail 154. The first rail 152 and the second rail 154 may be elongated sections of the handle 106 extending longitudinally in a proximal to distal direction. The first and second rails 152 and 154 may extend the length of the slot 120, e.g., proximally to just distal of the ring 116 and distally to proximal of the distal tip 200. As discussed below, the distalmost end of the main body 114a may include a keyed portion 178 that may be received into a proximal portion of a distal handle tip half 200a or 200b.

As discussed above, the first and second rails 152, 154 may define the slot 120 therebetween, extending longitudinally or in parallel through a portion of the main body 114 from a position spaced distally from the ring 116. The biasing element 122 may bias the spool 118 proximally within the slot 120.

Each main body half 114a, 144b includes a wire channel 176, an internal passage or groove. The wire channel 176 extends distally from the slot 120 to a distalmost end of the main body half 114a, 114b. The wire channel 176 extends longitudinally at a center of main body 114. The wire channels 176 of the main body halves 114a or 114b will encapsulate the wire 138 in a closed configuration of the handle 106. The wire channel 176 may have a shape and dimensions compatible with the size and shape of the wire 138.

As shown in FIG. 3, the first main body 114a and the second main body 114b may be identical, except that hinge components of the respective main body halves 114a and 114b may differ so that the main body halves 114a, 114b may be hingedly connected. The first main body 114a may include a hinge portion 146a that may be a hinge pin. The second main body 114b may include a hinge receptacle 148a that is an opening for the hinge portion 146a. During manufacture (prior to receipt of the medical device 100 by the user), the hinge portion 146a is coupled to the hinge receptacle 148a. The connection allows for a hinged assembly, including, for example, a hinge 150a, shown in FIG. 5. The main body 114 may include more than one hinge, and hinge 150a may include any suitable hinge structure allowing pivoting of main body halves 114a, 114b relative to one another to open and close the main body 114. The hinge 150a may be in any suitable position that permits the complete range of motion of the spool 118.

FIG. 3 shows a perspective view of the spool 118 that releasably attaches and translates relative to the main body 114 of the handle 106. The spool 118 may include two halves, 118a and 118b. FIG. 3 shows the halves 118a, 118b completely unattached for ease of view of the details of the halves 118a, 118b, but in embodiments, the halves 118a, 118b may be fixed together in a manner that permits opening and closing of the spool 118, such as by way of hinges. The division of the spool 118 into the halves 118a, 118b may be along a longitudinal plane at a center of the spool 118. The first spool half 118a and the second spool half 118b may be identical except for the coupling members of the hinges described below.

The first spool half 118a and the second spool half 118b may be placed onto main body halves 114a and 114b, respectively. In an embodiment, the spool half 118a may be placed over the rails 152 and 154 via slots or indentations 160a and 160b in an inner face of the spool half 118a, respectively. When the spool half 118a is placed on the rails 152 and 154, a portion of the spool half 118a may occupy a portion of the slot 120. The spool half 118b similarly is placed over the rails 152, 154 of the main body half 114b. The indentations 160a, 160b are designed to match the shape and size of the rails 152 and 154, with enough clearance for the spool 118 to slide on the rails 152, 154.

As shown in FIG. 3, the first spool half 118a and the second spool half 118b may be identical, except the hinge components of the respective spool halves 118a and 118b may differ so that the spool halves 118a, 118b may be hingedly connected. The first spool half 118a may include hinge portions 128a and 129a that may be hinge pins. The second spool half 118b may include hinge portions 128b and 129b that may be hinge receptacles. The hinge receptacles 128b, 129b may be openings for the hinge portions 128a, 129a. During manufacture, the hinge portions 128a, 129a are coupled to the hinge receptacles 128b, 129b. The connection allows for a hinged assembly, including, for example, hinges 130a and 130b, shown in FIG. 5.

FIG. 3 shows a perspective view of the distal handle tip 200 that releasably attaches to the main body 114 of the handle 106. The distal handle tip 200 may include two halves, 200a and 200b. FIG. 3 shows the halves 200a, 200b completely unattached for ease of view of the details of the halves 200a, 200b, but in embodiments, the halves 200a, 200b may be fixed together in a manner that permits opening and closing of the distal handle tip 200, such as by way of hinges. The division of the distal handle tip 200 into the halves 200a, 200b may be along a longitudinal plane at a center of the distal handle tip 200. The first handle tip half 200a and the second handle tip half 200b may be identical except for the coupling members of the hinges described below.

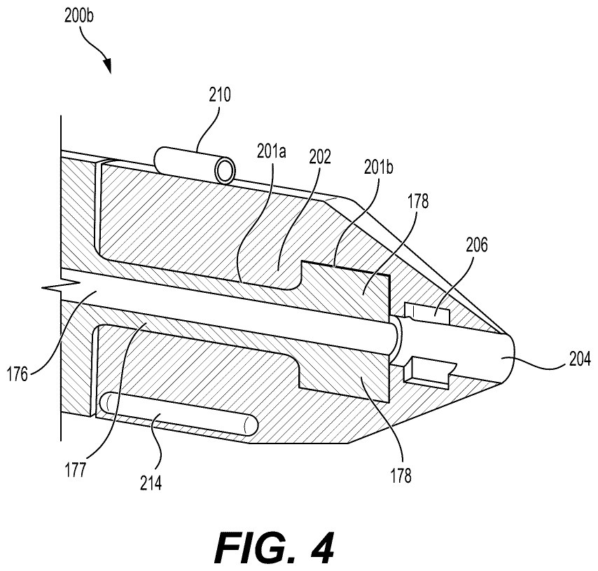

As shown in more detail in FIG. 4, the handle tip half 200b may include a central longitudinal proximal slot 201a extending distally from a proximal end of the tip half 200b. An indentation 202 of the tip half 200b defines the proximal slot 201a. The proximal slot 201a may have a semicircular cross-sectional shape. A distal end of the proximal slot 201a communicates with a distal slot 201b that has a semicircular cross-section that is wider than a cross-section of the proximal slot 201a. The slots 201a and 201b accept, respectively, a neck portion 177 and the keyed portion 178 of the main body half 114b. Each of the neck portion 177 and the keyed portion 178 has a semi-circular cross-sectional shape to match the shapes of the slots 201a, 201b, so that the distal tip 200 rotates relative to main body 114, which will rotate the shaft 108 relative to the wire 138.

Distal to the indentation 202, the distal handle tip half 200b may include a tapered distal portion with a cross-sectional width that decreases distally. The distal handle tip half 200b may taper to a narrow point and have a pointed or rounded shape at its most distal end.

As shown in FIG. 3, the distal handle tip half 200a and the distal handle tip half 200b may be identical, except the hinge components of the respective distal handle tip halves 200a and 200b may differ so that the main body halves 200a, 200b may be hingedly connected. The distal handle tip half 200a may include a hinge portion 208 that may be a hinge pin. The distal handle tip half 200b may include a hinge receptacle 210 that is an opening for the hinge portion 208. During manufacture, the hinge portion 208 is coupled to the hinge receptacle 210. The connection allows for a hinged assembly, including, for example, the hinge 212, shown in FIG. 5. The distal handle tip 200 may include more than one hinge, and the hinge 212 may include any suitable hinge structure allowing pivoting of the distal tip halves 200a, 200b relative to one another to open and close the distal handle tip 200.

As an alternative to or in combination with the distal hinge 212, a distal tip protrusion 214 may be positioned on the distal handle tip half 200a or 200b, as seen in FIG. 4. The distal tip protrusion 214 may be a raised or extended portion on one of the handle tip halves 200a or 200b designed to fit into a corresponding recess or indentation (not shown) on the other handle tip half. When the handle tip halves 200a and 200b are aligned and pressed together, the distal tip protrusion 214 may fit into the indentation in a friction fit fashion, creating a more secure closed configuration. The halves of the spool 118 and the main body 114 may also include similar mating protrusions and recesses that friction fit to create a more secure closed configuration.

The shaft 108 and the wire 138 may engage with additional features of the main body 114, the spool 118, and the distal handle tip 200. As discussed, the shaft 108 may extend from the distal portion 104 and radially surround the wire 138. A user may insert the shaft 108 and the wire 138 into main body halves 114a or 114b, the spool halves 118a or 118b, and the distal handle tip halves 200a or 200b, when these components are in an open configuration shown in FIG. 3. It is noted that FIG. 3 shows the half portions of each structure separated (unhinged), but it is understood that the open configuration of each handle component includes the respective halves pivotally attached at the hinges.

The wire 138 may occupy the slot 120 and be placed into the wire opening 119 of a spool half 118a or 118b. As shown in FIG. 3, the wire 138 may include a wire crimp 139 on its proximal-most end that may be inserted in the crimp slot or crimp receptacle 123. The crimp slot 123 may hold the wire crimp 139 when the wire 138 is fully inserted into the wire opening 119. The crimp slot 123 and the wire crimp 139 may have matching shapes, including noncircular shapes to prevent rotation of the wire 138 relative to the spool 118.

Each spool half 118a, 118b may be symmetric about a plane perpendicular to a longitudinal axis of the handle 106 and extending through a midpoint of the spool half 118a, 118b. That symmetry, along with a wire opening 119 and a crimp receptacle 123 at each end of each spool half 118a, 118b, allows a user to position each spool half 118a, 118b onto the main body 114 with either end of each spool half 118a, 118b facing distally.

A shaft opening 204 may accept the proximal end of the shaft 108. The shaft opening 204 may be at the distalmost end of the distal tip 200. As seen in FIG. 3, the shaft 108 may include a square (or otherwise noncircular) crimp 109 at a proximal end of the shaft 108. As seen in more detail in FIG. 4, a square (or otherwise matching noncircular cross section) recess 206 may be adjacent to the shaft opening 204. A user may insert the square crimp 109 into the square recess 206, where the recess 206 may assist in holding or fixing the shaft 108 and preventing its rotation relative to the distal tip 200.

An exemplary method for a user to assemble the medical device 100 will now be described. Prior to use, the main parts of the medical device 100 may be separate, uncoupled from one another. These parts include three discrete parts comprising the proximal component (the spool 118, the main body 114, and the distal handle tip 200) and the assembled distal component (the wire 138, the shaft 108, and all of the parts at the distal portion 104, including the end effector 110 and the coupling portion 132). Each of the separate, uncoupled proximal parts may be in an open configuration, as illustrated in FIG. 3 (except hingedly connected), before use. A user may place a first spool half 118a (or 118b) onto the rails 152 and 154 of a first main body half 114a (or 114b) until the rails 152 and 154 are fully within the indentations 160a, 160b. A user may then insert the wire 138 and the wire crimp 139 into the wire opening 119 and the crimp slot 123 of the spool half 118a, and the wire 138 into the wire channel 176 of the first main body half 114a. The other spool half 118b may then be closed onto the spool half 118a, via the hinges 130a, 130b, to secure the wire 138 therein.

As a next step, the user then engages one of the two distal tip halves 200a (or 200b) with the main body half 114a. The user may fix the indentation 202 to the keyed portion 178 of the main body half 114a, or otherwise place the neck 177 and the keyed portion 178 into slots 201a and 201b. Once the distal tip half 200a is fixed to the main body half 114a, the shaft crimp 109 is inserted into the recess 206, and the shaft 108 is placed in shaft opening 204. The other main body half 114b may be closed onto the main body half 114a, and the distal tip half 200b may be closed onto the distal tip half 200a, via their respective hinges, to transition the handle 106 to the closed configuration in FIGS. 1 and 5. The user may then perform a procedure with the medical device 100. In the embodiment, the shaft 108 may rotate relative to the wire 138, by rotating distal tip 200 relative to main body 114.

After the procedure is completed, the user may disassemble the medical device 100 by reversing the assembly steps described above. The user may then discard the distal components, which may have contacted internal parts of a patient. The proximal components can undergo a cleaning operation and reused in a subsequent procedure with sterile, new distal components. Although the description above depicts and describes a particular order of method steps, it will be appreciated that some or all of the steps above may be repeated or performed in alternative orders.

In another embodiment, the distal tip 200 may be omitted from the handle. The main body 114 may include at least some of the features of the distal tip 200. For example, the main body 114 may include the shaft opening 204 and the recess 206. The shaft opening 204 and the recess 206 may be at the distalmost end of the main body 114. The exemplary method of assembly in the embodiment of FIGS. 1-5 above may be used in this embodiment, except that a distal tip 200 is not engaged to the main body 114. For example, a user may place a first spool half 118a (or 118b) onto the rails 152 and 154 of a first main body half 114a (or 114b) until the rails 152 and 154 are fully within the indentations 160a, 160b. A user may then insert the wire 138 and the wire crimp 139 into the wire opening 119 and the crimp slot 123 of the spool half 118a, and the wire 138 into the wire channel 176 of the first main body half 114a. Once the spool half 118a is fixed to the main body half 114a, the shaft crimp 109 is inserted into a recess of the main body, and the shaft 108 is placed in a shaft opening of the main body (not shown). The other main body half 114b may be closed onto the main body half 114a via their respective hinges. Then, the other spool half 118b may be closed onto the spool half 118a, via the hinges 130a, 130b, to secure the wire 138 therein and transition the handle 106 to the closed configuration in FIGS. 1 and 5.

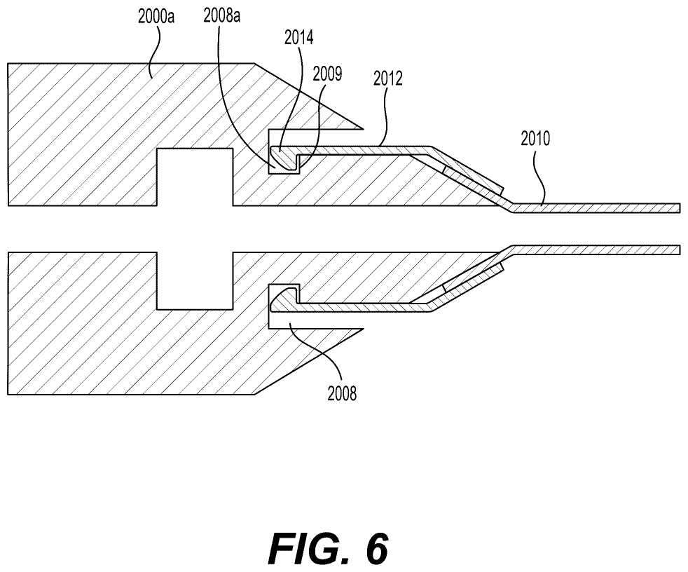

FIG. 6 illustrates another embodiment of a medical device including a different structure for coupling a shaft 2010 to a distal tip. The distal handle tip may include two identical halves, only one of which (distal tip half 2000a) is shown in FIG. 6. Except as shown and described here, the distal tip halves of this embodiment are identical to the distal tip halves 200a, 200b in the embodiment in FIGS. 1-5.

In the embodiment shown in FIG. 6, the shaft 2010 may include a proximal end that is flared outwardly. A collar 2012 may surround an outside of the flared portion of the shaft 2010. The collar 2012 may be annular and flexible, such as a flexible tubing. In addition, or alternatively, the proximal side of the collar 2012 may include longitudinal slits therethrough to define circumferentially-arranged, longitudinal fingers that can flex radially inward and outward. Each finger may include a radially-inward directed protrusion 2014.

The collar 2012 may move relative to the shaft 2010 (rotate and translate) and transition in and out of a slot 2008 in the distal tip halves (including half 2000a). The slot 2008 may be annular and have an outer diameter that is greater than an outer diameter of the collar 2012. The slot 2008 accepts the collar 2012. Upon full insertion of the collar 2012 into the slot 2008, the protrusion 2014 sits in a corresponding annular opening 2008a of the slot 2008. In this way, the protrusion 2014 is proximal of a ledge 2009 that prevents removal of the collar 2012 from the distal tip unless sufficient removal force is applied. As the collar 2012 enters the slot 2008, the proximal portion of the collar 2012 (for example the fingers described above) flex radially outward and the protrusion rides on an inner surface of the slot 2008. Once the collar 2012 is fully inserted, the proximal portion of the collar 2012 (for example the fingers described above) attain the position shown in FIG. 6.

The exemplary method of assembly in the embodiment of FIGS. 1-5 may be used in this embodiment, except as shown and described here. For example, a user may assemble the spool 118, the main body 114, insert the wire 138 into the spool 118, and engage the distal tip to the main body 114 in the same manner as described above. However, the insertion of the shaft 2010 differs. Instead, in this embodiment, the collar 2012 may slide over the shaft 2010 and be introduced into the slot 2008 of the distal tip until the protrusion 2014 enters the annular opening 2008a. After the procedure is completed, the user may disassemble the medical device by reversing the assembly steps described above.

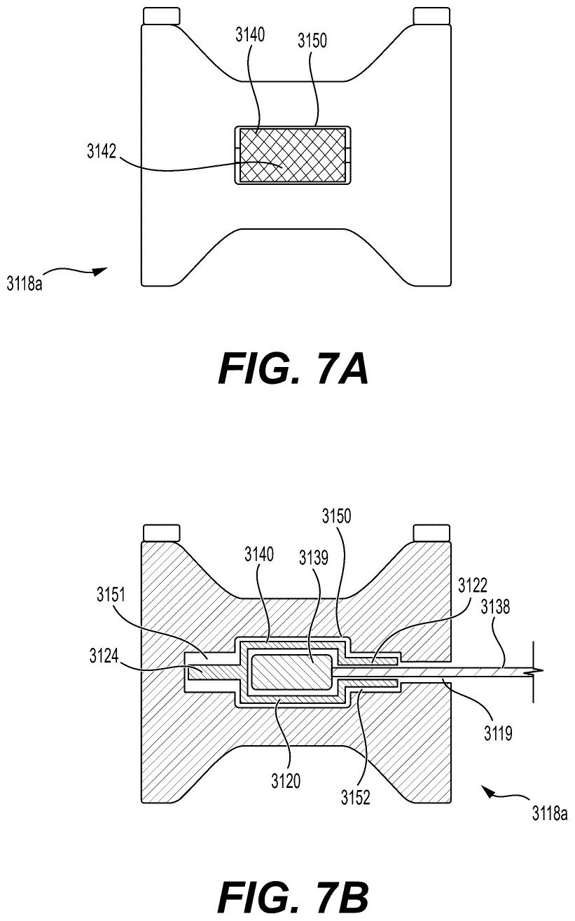

FIGS. 7A-B illustrate another embodiment of a medical device, including an arrangement that permits rotation of the actuation wire relative to the handle. This rotation causes rotation of the end effector relative to the shaft. FIGS. 7A and 7B show an actuator that may be a spool half 3118a, in addition to a knob 3140. A spool may include two halves, 3118a and a second half identical to half 3118a except for the different hinge parts. These spool halves (including spool half 3118a) may share the same features of the spool halves 118a and 118b in FIGS. 1-5, except as described herein.

FIG. 7A shows the radially outer side of the spool half 3118a that a user engages during use. The spool half 3118a includes a central opening 3150 through the thickness of the spool half 3118a. A knob 3140 is accessible through the opening 3150 and rotates relative to the opening 3150. The outer side of the knob 3140, accessible by the user, may include a textured/knurled surface 3142 for a user to more easily rotate the knob 3140. The central opening 3150 may be on either or both of the spool halves.

FIG. 7B shows the inner side of the spool half 3118a, the side on which the other spool half closes, and cross sectional portions of parts siting in the spool half 3118a. The inner side of the spool half 3118a defines four slots or openings that communicate with one another, namely (and in order from proximal to distal) a proximal slot 3151, the central opening 3150, a distal slot 3152, and a wire opening 3119. Of these, only the central opening 3150 extends radially through the entire thickness of the spool half 3118a. As shown in FIG. 7B, the proximal and distal slots 3151, 3152 have smaller widths than the central opening 3150, and the wire opening 3119 has a smaller width than the distal slot 3152.

The knob 3140 sits within the proximal slot 3151, the central opening 3150, and the distal slot 3152. The knob 3140 includes first and second protrusions 3122 and 3124 that sit within the distal slot 3152 and the proximal slot 3151, respectively. The central portion of the knob 3140 defines a crimp opening or slot 3120 and is positioned within the central opening 3150, at a midpoint along the length of the spool half 3118a. The first protrusion 3122 of the knob 3140 includes a central channel aligned with the wire opening 3119. Although not shown, the wire opening 3119 may be found on either side of the spool (see, e.g., FIG. 3 opening 119 on spool halves 118a, 118b).

The wire 3138 may be introduced into the wire opening 3119 of the spool half 3118a. As shown in FIG. 7B, the wire 3138 may include a rectangular wire crimp 3139 on its proximal-most end that may be inserted in the crimp slot 3120. The crimp slot 3120 may hold the wire crimp 3139 when the wire 3138 is fully inserted into the wire opening 3119. The crimp slot 3120 and the wire crimp 3139 may be rectangular or otherwise non-circular. The knob 3140 surrounds a portion of the wire 3138 and the crimp 3139, such that the knob 3140 engages with the crimp 3139 to help rotate the crimp 3139 and the wire 3138 as the knob 3140 rotates.

In these aspects, rotation of knob 3140 (e.g., clockwise or counterclockwise) also rotates the wire 3138 and thus also an end effector that may be at a distal end of the distal portion of the medical device. For example, the user may rotate knob 3140 (e.g., relative to the opening 3150 about a central longitudinal axis of the knob 3140) to rotate the wire 3138 (via the crimp 3139) and the end effector to orient or otherwise position the end effector at the treatment site. In these aspects, the user may rotate the knob 3140 clockwise, which also rotates the wire 3138 and the end effector clockwise. Similarly, the user may rotate the knob 3140 counterclockwise, which also rotates the wire 3138 and the end effector counterclockwise.

The exemplary method of assembly of the embodiment of FIGS. 1-5 may be applied in this embodiment, except as shown and described here. A user may still begin by placing a first spool half 3118a (or the other spool half) onto the rails 152 and 154 of a first main body half 114a (or 114b) until the rails 152 and 154 are fully within the indentations 160a, 160b. The user may then insert the wire crimp 3139 into the crimp slot 3120 and the wire 3138 into the wire opening 3119 of the spool half 3118a. The next steps remain the same, and after the procedure is completed, the user may disassemble the medical device by reversing the assembly steps described above.

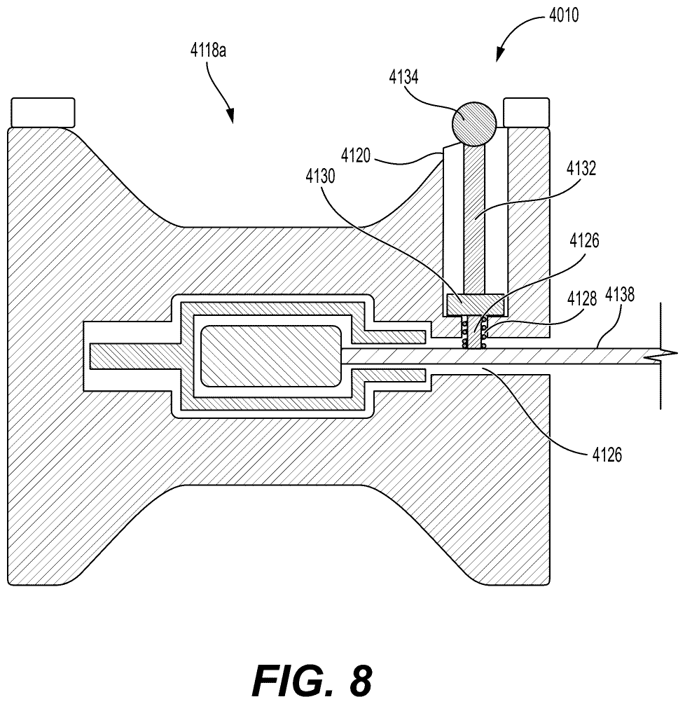

FIG. 8 illustrates another embodiment of a spool assembly similar to that of FIGS. 7A-7B, except with an electrocautery connection. This connection may apply to any of the described embodiments. A spool may include two halves, 4118a and another half not shown. These spool halves may have any of the features of the spool halves 118a, 118b of FIGS. 1-5 or the spool half 3118a in FIGS. 7A-7B, except for the electrocautery connector 4010 described below.

The electrocautery connector 4010 is electrically couples the wire 4138 to an electrosurgical generator (not shown). The connector 4110 is positioned within a radial slot 4120 at the distal-most end of the spool 4118. The connector 4110 may include a pin-shaped distal coupler 4126, a spring 4128, a stop 4130, a cord 4132, and a plug 4134.

The plug 4134 may couple to an energy source that generates an electrical current. The cord 4132 may couple to the inferior side of the plug 4134, and the cord may couple to the top of the stop 4130. The spring 4128 may rest against the bottom of the stop 4130 and surround the coupler 4126. The wire 4138 and the coupler 4126 are in constant electrical contact, without limiting the rotation of the wire 4138. The electrical connection sends electrical currents distally from the plug 4134 (connected to the electrical source) to the wire 4138 to the distal portion of the medical device. The end effector may receive the current and be used as an electrode that may, for example, cut or cauterize tissue.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered limited by the foregoing description.

We claim:

1. A medical device, comprising:
a handle including a main body and an actuator;
wherein the main body includes a first portion and a second portion, and the main body has an open configuration and a closed configuration; and
wherein the actuator includes a first portion and a second portion, the actuator has an open configuration and a closed configuration, and the actuator translates relative to the main body in the closed configurations of the main body and the actuator.

2. The medical device of claim 1, wherein the first portion of the main body pivots relative to the second portion of the main body to transition from the open configuration to the closed configuration.

3. The medical device of claim 1, wherein each of the first portion and the second portion of the main body includes a first rail and a second rail, wherein each of the first rail and the second rail extends longitudinally in a proximal to distal direction and define a slot therebetween.

4. The medical device of claim 1, wherein each of the first portion of the main body and the second portion of the main body includes a wire channel extending longitudinally.

5. The medical device of claim 3, wherein each of the first portion of the actuator and the second portion of the actuator includes indentations for placing over the first rail and the second rail.

6. The medical device of claim 1, wherein the first portion of the actuator pivots relative to the second portion of the actuator to transition from the open configuration to the closed configuration.

7. The medical device of claim 1, wherein the handle includes a distal tip, wherein the distal tip includes a first portion and a second portion, and the distal tip has an open configuration and a closed configuration.

8. The medical device of claim 7, wherein each of the first portion of the distal tip and the second portion of the distal tip defines a proximal slot proximal of and communicating with a distal slot having a cross-section that is wider than a cross-section of the proximal slot.

9. The medical device of claim 8, wherein the proximal slot and the distal slot of each of the first portion of the distal tip and the second portion of the distal tip respectively receives a neck portion and a keyed portion of each of a corresponding first portion of the main body and the second portion of the main body, wherein each keyed portion has a cross-sectional shape to match a shape of a corresponding distal slot.

10. The medical device of claim 7, wherein the first portion of the distal tip is pivotally connected to the second portion of the distal tip to transition between the open configuration and the closed configuration of the distal tip.

11. The medical device of claim 7, wherein a shaft extends from a distal end of the handle and radially surrounds a wire, wherein the shaft includes a shaft crimp, and wherein the wire includes a wire crimp, wherein the wire crimp is positionable in the actuator, and the shaft crimp is positionable in the distal tip.

12. The medical device of claim 11, wherein a first end of the actuator includes a first wire opening and a first crimp slot, and a second end of the actuator includes a second wire opening and a second crimp slot.

13. The medical device of claim 11, wherein the shaft includes a proximal portion that is flared outwardly, and the medical device includes a collar surrounding the flared proximal portion of the shaft, wherein the distal tip includes a slot, and wherein the collar is receivable into the slot.

14. The medical device of claim 1, wherein the actuator includes an opening extending radially through the actuator, wherein the actuator includes a knob that rotates within the opening.

15. The medical device of claim 14, wherein the knob defines an opening therein to receive a wire.

16. A medical device, comprising:
a proximal component, including a handle having a main body and an actuator;
wherein the main body includes a first portion and a second portion movable relative to the first portion of the main body to transition between an open configuration and a closed configuration of the main body;
wherein the actuator includes a first portion and a second portion movable relative to the first portion of the actuator to transition between an open configuration and a closed configuration of the actuator, and the actuator translates relative to the main body in the closed configurations of the main body and the actuator; and
a distal component including a shaft and a wire, wherein the distal component is releasably coupled to the proximal component.

17. The medical device of claim 16, wherein the proximal component includes a distal tip, wherein the distal tip includes a first portion and a second portion movable relative to the first portion of the distal tip to transition between an open configuration and a closed configuration of the distal tip.

18. The medical device of claim 17, wherein the shaft extends from a distal end of the proximal component and radially surrounds the wire, wherein the shaft includes a portion that is flared outwardly, and the medical device further comprises a collar that surrounds the flared portion of the shaft, wherein the distal tip includes a slot, and wherein the collar of the shaft is receivable into the slot.

19. The medical device of claim 16, wherein the actuator includes an opening extending radially through the actuator, wherein the actuator includes a knob that rotates within the opening, and wherein the knob defines an opening therein to receive a wire.

20. A method of assembling a medical device, comprising:
placing a first actuator portion onto a first main body portion;
inserting a wire into a wire opening of the first actuator portion and a wire channel of the first main body portion;
closing a first main body portion with a second main body portion, wherein the first main body portion and the second main body portion are configured to be closed by pivoting the first main body portion relative to the second main body portion; and
closing the first actuator portion with a second actuator portion, wherein the first actuator portion and the second actuator portion are configured to be closed by pivoting the first actuator portion relative to the second actuator portion.
